(19) United States
(12) Patent Application Publication
(10) Pub. No.: US 2008/0300322 A1
(43) Pub. Date: Dec. 4, 2008

(54) Delivery vehicles containing rosin resins

(75) Inventors: Atul J. Shukla, Cordova, TN (US); Anthony Edward Soscia, Atlanta, GA (US); Yingxu Peng, Pennington, NJ (US); Yichun Sun, Gaithersburg, MD (US); James R. Johnson, Germantown, TN (US)

Correspondence Address: HOWARD EISENBERG, ESQ., 1220 LIMBERLOST LANE, GLADWYNE, PA, 19035, US

(73) Assignee: Atlantic Pharmaceuticals, Inc.

(21) Appl. No.: 11/809,839

(22) Filed: Jun. 1, 2007

(51) Int. Cl. A61K 47/06; A61P 43/00

(52) U.S. Cl. 514/782

(57) ABSTRACT

Compositions containing a vehicle containing a rosin resin or derivative and a non-volatilized solvent or plasticizer. The compositions may be used to deliver a biologically active substance such as an active pharmaceutical ingredient. The concentration of rosin resin or derivative in the vehicle is sufficient to alter the release rate from the composition of a biologically active substance that is dissolved, dispersed, emulsified, or suspended in the vehicle.

### FIELD OF THE INVENTION

The invention pertains to vehicles for delivery of biologically active substances (BAS), such as active pharmaceutical ingredients (APIs). In a particular embodiment, the invention pertains to vehicles, and to compositions containing the vehicles, for controlled release of an active pharmaceutical ingredient to a living organism.

### BACKGROUND OF THE INVENTION

Delivery vehicles, often referred to by the synonymous term “vehicle”, are essential components of formulations for administration of biologically active substances (BAS), such as active pharmaceutical ingredients (APIs). The BAS is typically dissolved, dispersed, emulsified or suspended in the delivery vehicle to form a solution, a matrix, a reservoir, an emulsion, or a suspension. The BAS and the delivery vehicle may be combined in the form of a pellet, bead, granule, rod, mass of various shapes and sizes, or microparticles. The BAS-loaded vehicle may be formed into various shapes in order to fit inside of a cavity, such as those that exist within the body of an organism. The vehicle and BAS may also be granulated and pressed into a tablet via conventional pharmaceutical methods or formed into a bead or pellet and placed within a capsule for oral application. The physical form of a BAS-loaded vehicle may be a liquid, a gel, a semisolid, a paste, a film or a solid.

A composition containing an API and a delivery vehicle may be administered to a subject through various routes, such as by oral, topical, rectal, vaginal, surgical implantation or parenteral routes. The physiochemical properties of the vehicle, such as hydrophobicity/hydrophilicity, viscosity, geometric shape, and amount of the vehicle, are important in determining the release characteristics of the administered API from a vehicle and from a dosage form containing the vehicle and the API. Typically, hydrophilic delivery vehicles rapidly release the API into the body. In contrast, hydrophobic vehicles typically retard the contact of an API with aqueous body fluids and hence can be used to slow the release of an API from the site of administration. For many administration indications, such controlled release of an API from the site of administration is desired.

A desirable characteristic of a vehicle is that its properties, such as viscosity, consistency, shape, and degree of hydrophilicity/hydrophobicity, should be capable of being tailored. For example, a vehicle may be formed or pressed into shapes and implanted into a body. Additionally, a vehicle loaded with an API may be microencapsulated, formed or compressed into tablets or placed into hard-shell capsules for oral ingestion or for implantation into a body cavity. Further, an ideal vehicle should be capable of being tailored to provide a range of viscosity, consistency, hydrophilicity, and hydrophobicity. Modification of vehicle properties such as these permits a formulator to produce a composition having desired API release characteristics, such as delayed, extended, pulsatile, or controlled release.

The following patents, each of which is incorporated herein by reference, disclose delivery vehicles for controlled delivery of BAS. Maulding, U.S. Pat. No. 4,297,353, discloses a glyceride vehicle comprising a glycerol ester of a vegetable fatty acid. Carlsson, U.S. Pat. No. 6,117,857, discloses a vehicle of an admixture of a galactolipid extracted from plant material and a polar solvent, such as water, glycerol, ethanol, propylene glycol, polyethylene glycol, polypropylene glycol, glycofurol, N-methylpyrrolidone, or TRANSCUTOL. Brooks, U.S. Pat. No. 5,352,662, discloses a vehicle for extended release formulations comprising a biocompatible hydrophobic vehicle such as sesame seed oil and a polyglycerol ester such as diglycerol tetrastearate. Tipton, U.S. Pat. No. 5,747,058, discloses a high viscosity liquid controlled delivery system (HVLCM) in which the vehicle is a combination of a viscous liquid vehicle, such as sucrose acetate isobutyrate (SAIB), and a solvent, which may be ethanol, dimethylsulfoxide, ethyl lactate, benzyl alcohol, triacetin, 2-pyrrolidone, N-methylpyrrolidone, propylene carbonate, or glycofurol. Wicks, U.S. Pat. No. 6,001,822, discloses an antiparasitic formulation in a vehicle of 50% to 95% sesame oil, with the remainder ethyl oleate. Hausheer, U.S. Pat. No. 5,958,937, discloses a vehicle for producing a formulation of poorly water-soluble camptothecin and its analogues. The vehicle of Hausheer is N-methyl-2-pyrrolidone with additives such as surfactants, polyethylene glycol, ethyl alcohol, and benzyl alcohol. Shukla et al., U.S. Pat. No. 6,960,346, describes the use of citric acid esters (CAE) such as triethyl citrate (TEC), acetyl triethyl citrate (ATEC), tributyl citrate (TBC), acetyl tributyl citrate (ATBC), acetyltri-n-hexyl citrate (A-6), and butyryltri-triethyl n-hexyl citrate (B-6) as vehicles for delivery of biologically active substances.

These and other presently available vehicles, while providing certain desirable characteristics for a delivery vehicle, fail to provide optimal characteristics of a delivery vehicle. For example, the stability of an API is often compromised in the presence of vegetable oils. Vehicles such as sucrose acetate isobutyrate (SAIB) and citric acid esters (CAE) are soluble in low molecular weight alcohols and exist primarily as liquids at room temperature. Liquid vehicles may be unsuitable for formulating into solid dosage forms such as tablets, microcapsules, or implantable dosage forms. Therefore, a significant need exists for a vehicle that overcomes the disadvantages of the currently available vehicles and that may be used to provide compositions that are suitable for delivery of a wide variety of APIs using various dosage forms or delivery platforms and that may be used to tailor the rate of delivery of the API.

Resin from which the volatile terpene components have been removed by distillation is known as rosin. Rosin is insoluble in water and is thermoplastic in its solid form. Rosin is typically composed of a complex mixture of different substances including organic diterpene acids, often referred to as resin acids. These are closely related to the terpenes, and are derived from terpenes through partial oxidation. Resin acids may be dissolved in alkalis to form resin soaps, from which the purified resin acids are regenerated by treatment with acids. Examples of resin acids are abietic acid (sylvic acid), C₂₀H₃₀O₂, plicatic acid contained in cedar, and pimaric acid, C₂₀H₃₅O₂, a constituent of gallipot resin. The principal rosin acid is abietic acid. Abietic acid can also be extracted from rosin by means of hot alcohol. It crystallizes in leaflets, and on oxidation yields trimellitic acid, isophthalic acid and terebic acid. Abietic acid is often used as a representative structure for “rosin”, but in fact, rosin is made up of more than 20 isomeric resin acids in the naturally occurring rosin substance. Abietic acid is often the largest component (wt %) among the various resin acids in rosin. Under certain conditions such as heat and acid exposure, the resin acids in rosin may form dimers.

Rosin, also known as colophony, is usually named according to the acquisition source. For example, gum rosin is derived from the oleoresin (pine gum) exudate collected of living trees. The harvesting of the oleoresin is simple, involving only periodic wounding of the tree and collecting the exudate. Wood rosin is derived from the stump wood of the tree and tall oil rosin is obtained by distillation of crude tall oil. Rosin has poor stability caused by unsaturation. However, stability can be improved by various modifications such as disproportionation and hydrogenation.

Rosin resin derivatives are typically produced by the esterification of rosin, hydrogenated rosin, or polymerized rosin with a polyhydric alcohol such as glycerol or pentaerythritol. Modification of rosin resin may reduce the acid number, alter the softening point, improve the chemical stability, improve the oxidative stability, alter the polarity, and modify the volatility. Rosin esters are sometimes referred to as hard resins or synthetic resins.

Rosin resin and its derivatives, referred to herein as “RR&D”, have a history of use as excipients in the manufacture of topical delivery systems and oral pharmaceutical dosage forms, and as ingredients in chewing gums, in which rosin resins are utilized as softening and plasticizing agents, and in beverages. In topical delivery systems, RR&D are used as components in adhesives. RR&D are also used in the preparation of oral dosage forms, for example as tablet binding agents, as drug releasing films, or as components in microparticles and matrices.

Patents disclosing the use of RR&D include the following: Klofta, U.S. Patent. Application No. 2004/0191279, lists rosin resins such as those derived from gum, wood and tall oil rosin as adhesives for topical delivery systems. Fenton et al., U.S. Pat. No. 3,959,510, describes the use of rosin resin as a beverage clouding agent. Koch et al, U.S. Pat. No. 4,187,320; Teng et al., U.S. Pat. No. 3,883,666; Chuu, et al., U.S. Pat. No. 5,087,459; and Anderson et al., U.S. Pat. No. 5,487,902, describe the use of rosin resin derivatives, such as glycerol ester of polymerized rosin, pentaerythritol ester of wood or gum rosin, pentaerythritol ester of partially hydrogenated wood or gum rosin, glycerol ester of partially hydrogenated wood or gum rosin, for delivery of active agents in a medicated chewing gum. Andersen also discloses the use of a masticated chewing gum base containing rosin resin, which allows for delayed release of fat soluble active ingredients. Medicated chewing gum requires the input of physical energy (mastication) in order to liberate the API contained therein, in contrast to the compositions of the invention as disclosed below, and is typically limited to the non-regulated delivery of an API lasting only a few hours.

As discussed below, the present invention concerns the control of release of a BAS from a composition by utilizing a combination of an RR&D and a solvent. Accordingly, dosage forms that require the input by an individual of physical energy produced by muscles controlled by the somatic nervous system in order to control the release of an API from the dosage form, such as a medicated gum from which the release of the medication is controlled by the conscious act of chewing following administration of the gum into the mouth, are expressly excluded from the scope of the present invention.

RR&D have been used as hydrophobic matrix materials. (Pathak and Dorle, Rosin and rosin derivatives as hydrophobic matrix materials for controlled release of drugs, Drug Design and Delivery, 6:223-227 (1990)). In the Pathak and Dorle article, granule matrices of an RR&D and aspirin were fabricated using acetone in a volatilized solvent evaporation technique to make the granules, which were subsequently compressed into tablets. The volatilized acetone solvent was utilized as a processing aid and did not form a part of the final formulation containing the rosin derivative and the aspirin. The tablets made with this method did not sustain drug release for more than 8 hours.

Rosin resins have also been disclosed in the manufacture of drug-containing nanoparticles. (Lee et al., Rosin nanoparticles as a drug delivery carrier for the controlled release of hydrocortisone, Biotechnology Letters, 27:1487-1490 (2005)). Lee disclosed the preparation of nanoparticles with a particle size ranging from 167-332 nm containing a physical mixture of rosin and hydrocortisone. The nanoparticles were prepared using a volatilized organic solvent evaporation system and less than 50% of the drug was released within 24 hours in simulated biological fluid.

RR&D have also been used to form films for coating drug-containing particles. (Satturwar et al., Evaluation of new rosin derivatives for pharmaceutical coating, International Journal of Pharmaceutics, 270:27-36 (2004)). Satturwar coated drug containing particles with rosin derivatives and obtained a sustained drug release from the particles for a period of 10 hours. RR&D polymers have also been studied as implant matrix films containing drug. (Fulzele et al., Novel Biopolymers as Implant Matrix for the Delivery of Ciprofloxacin: Biocompatibility, Degradation, and In Vitro Antibiotic Release, J Pharm Sci 96:132-144 (2006)).

The above films were made by solvent casting. Such films are difficult to manufacture on a commercial scale and are typically not designed to hold a large capacity of BAS. The solvent casting method of manufacture of these delivery systems utilizes a volatilized organic solvent for the partial or full solubilization of the rosin resin or its derivative and the API in order to form an API in a rosin resin matrix. The volatilized solvent in these systems is completely or almost completely evaporated out of the composition leaving behind a mixture from which the final dosage form is manufactured. In this type of system, the volatilized organic solvents are utilized as processing aids and are not components of the vehicles. The removal of the solvent from these prior art compositions limits the usefulness of the RR&Ds in the number and type of dosage forms that may be prepared and in the release characteristics of the API in the final dosage forms. Thus, compositions in the form of a film, made by solvent casting or other method in which essentially all of the solvent for a BAS is removed, are expressly excluded from the scope of the present invention.

### BRIEF DESCRIPTION OF THE DRAWINGS

FIG. 1 is a graph showing the percentage of BAS (guaifenesin) released from compositions containing vehicles containing an RR&D (glycerol ester of wood rosin) and an NVS/P (TEC) versus time.

FIG. 2 is a graph showing the percentage of BAS (guaifenesin) released from compositions containing vehicles containing varying concentrations of an RR&D (glycerol ester of wood rosin) and a combination of NVS/Ps (TEC, PEG 6000) versus time.

FIG. 3 is a graph showing the percentage of BAS (guaifenesin) released from compositions containing vehicles containing a RR&D (glycerol ester of wood rosin) and a combination of NVS/Ps (TEC and PEG; TEC, ATEC, and PEG, or ATEC and PEG) versus time.

FIG. 4 is a graph showing the percentage of a BAS released from compositions containing a multiplicity of BAS (guaifenesin and hydrocodone) and vehicles containing RR&D (glycerol ester of wood rosin) and a combination of NVS/Ps (ATEC, and PEG) versus time.

FIG. 5 is a graph showing the percentage of a BAS (hydrocodone) released from compositions containing a multiplicity of BAS (guaifenesin and hydrocodone) and vehicles containing RR&D (glycerol ester of wood rosin) and a combination of NVS/Ps (ATEC, and PEG) versus time.

FIG. 6 is a graph comparing the percentage release of two BAS (guaifenesin and hydrocodone) from compositions containing vehicles containing an RR&D (glycerol ester of wood rosin) and a combination of NVS/Ps (ATEC, and PEG) versus time.

FIG. 7 is a graph showing the release of a BAS (guaifenesin) from compositions containing an RR&D (glycerol ester of wood rosin) and two NVS/P (ATEC and PEG) and from similar compositions containing a single NVS/P (ATEC).

FIG. 8 is a graph showing the release of a BAS (guaifenesin) from a composition containing an NVS/P (TEC) and no RR&D and from compositions containing an NVS/P (TEC) and increasing concentrations of an RR&D (glycerol ester of wood rosin).

### DETAILED DESCRIPTION OF THE INVENTION

It has been discovered that the combination of a rosin resin or a derivative (RR&D) and a non-volatilized solvent and/or plasticizer (NVS/P) for use as a vehicle for a BAS overcomes many of the disadvantages inherent in presently available vehicles. Vehicles containing an RR&D and an NVS/P have been surprisingly discovered to be ideally suited for delivery of BAS such as active pharmaceutical ingredients (API), such as pharmacological agents, and as components in formulations and compositions, such as pharmacological formulations and compositions. The current inventors have discovered that incorporating and retaining one or more solvents and/or plasticizers (NVS/P) along with one or more rosin resins or derivatives (RR&D) allows for the formation of a novel delivery vehicle that provides dramatic improvements in modulating the release characteristics of a BAS, such as an API. With the vehicles and compositions of the invention, release of the BAS may range from up to 8 hours to 24 hours or longer. The vehicles and compositions of the invention further provide a wide variety of consistencies, hydrophobicities/hydrophilicities, and shapes for delivery of APIs, such as liquids, gels, pastes, beads, rods, cylinders, granules, tablets, capsules, or various shapes for delivery via topical, transdermal, oral, injectable, or implantable routes.

As used herein, the term “vehicle” is synonymous with the term “delivery vehicle” and refers to a compound or combination of compounds that functions as a diluent or carrier for a BAS.

As used herein, the term “composition” refers to a discrete physical entity containing one or more constituents. As an example, a composition may consist of a vehicle. As another example, a composition may contain a vehicle and a BAS. As another example, a composition may contain a vehicle, a BAS, and additional additives. As another example, a composition may be a particle containing an API and a vehicle, which composition may be encapsulated by a second composition containing additional amounts of the same or different API with or without additional components such as a binder. A composition may be solid, liquid, or semi-solid and may exist in a single phase, such as a solution, or in multiple phases, such as a suspension, dispersion, or emulsion. In the case of a multiple phase composition, although the combination of phases constitutes a single composition, the individual phases may also be considered to be distinct compositions.

As used herein, the term “formulation” refers to a dosage form that may be administered to an individual or to the environment. A formulation may contain a single composition, such as a single or multiphase composition, or may contain a multiplicity of compositions. Examples of formulations containing a multiplicity of compositions include multilayer tablets wherein each layer may be considered to be a separate composition, a coated particle of a composition wherein the coating and the particles may be considered to be separate compositions, a coated capsule wherein the capsule, with or without its contents, and the coating may be considered to be separate compositions, and a dispersion or emulsion wherein the internal and external phases of the dispersion or emulsion may be considered to be individual compositions.

As defined herein, the use of such a vehicle containing an RR&D and an NVS/P indicates that the amount of RR&D and NVS/P in the composition containing the vehicle and a BAS is sufficient to dissolve, disperse, emulsify, or suspend the quantity of BAS contained within the composition and the entire quantity of the BAS is dissolved, dispersed, emulsified, or suspended in the vehicle.

As used herein, the term “non-volatilized”, when referring to an NVS/P, means that at least 10% of the quantity (% w/w) of the NVS/P that was utilized in the formulation of a composition containing the non-volatilized NVS/P remains in the composition.

It has been discovered that formulations containing compositions that contain both an RR&D and an NVS/P as a vehicle for a BAS, such as an API, are ideal for the administration of a BAS. If used for delivery of an API to an organism, the RR&D selected should be non-toxic in the levels utilized, water insoluble, and compatible with the API with which it is formulated. The form of the vehicle prepared with a RR&D and a NVS/P may be tailored within a wide range to provide, for example, a liquid, a semisolid, a gel, a paste, or a solid.

In one embodiment, the invention is a delivery vehicle for a biologically active substance (BAS) comprising one or more rosin resin or derivatives (RR&D) and one or more non-volatilized solvents or plasticizers (NVS/P), wherein the concentration of the RR&D relative to the NVS/P in the delivery vehicle is sufficient to modify the rate of release from the delivery vehicle of a BAS that is dissolved, suspended, emulsified, or dispersed therein, as compared to the rate of release of the BAS from a similar delivery vehicle that is free of the RR&D.

In another embodiment, the invention is a composition, such as a BAS or API composition, comprising one or more BAS, such as an active pharmaceutical ingredient, one or more NVS/Ps combined with one or more RR&Ds, with or without a suitable pharmaceutical excipient or excipients. The quantities and ratios of the RR&D and the NVS/P in the composition are sufficient to dissolve, suspend, emulsify, or disperse the entire quantity of API contained in the formulation. The quantity of RR&D in the composition is sufficient to modulate the release characteristics of the API from the composition. That is, the release characteristics of the API from the composition of the invention will differ from that of a similar composition in which the vehicle lacks the RR&D.

In another embodiment, the invention is a formulation or dosage form for administration of a BAS, such as an API, to an individual in need thereof. The formulation of the invention contains one or more compositions, at least one of which is a composition of the invention that contains a BAS, such as an API, that is dissolved, suspended, emulsified, or dispersed in a vehicle containing one or more NVS/Ps and one or more RR&Ds.

In one particular preferred embodiment, the invention is a formulation containing a multiplicity of compositions, at least one of which is a composition of the invention containing a BAS that is dissolved, suspended, emulsified, or dispersed in a vehicle containing one or more NVS/P and one or more RR&D. The compositions of the formulation other than the composition of the invention may include a BAS that is the same as or different from the BAS that is contained within the composition of the invention within the formulation. The compositions of the formulation other than the composition of the invention may or may not contain a vehicle that includes an NVS/P and an RR&D. If the composition not of the invention within the formulation contains a vehicle that includes an NVS/P and an RR&D, then the concentration of the NVS/P and the RR&D is insufficient to completely dissolve, suspend, disperse, or emulsify the BAS in the composition or the amount of RR&D in the vehicle is insufficient to modulate the release rate of the BAS from that composition.

This type of formulation may be useful to provide a dosage form that delivers a loading dose of a BAS, such as from the composition of the formulation that is other than of the present invention, and that further delivers a BAS in a controlled release manner, such as from the composition of the invention that is contained within the formulation.

The compositions and formulations of the invention, as described more fully below, may have a variety of release characteristics, such as immediate, sustained, delayed, pulsatile, or controlled. More than one RR&D and NVS/P may constitute the vehicle, which may provide for sustained, delayed, pulsatile, or controlled release of the BAS. Additionally, the RR&D NVS/P vehicle may contain one or more additives that may modulate the release of the BAS, alter the viscosity or consistency of the composition, protect constituents of the composition or formulation from oxidation, provide a color or taste, or vary the hydrophobicity or hydrophilicity of the vehicle or the composition containing the vehicle and the BAS. As used herein, the term “controlled release” may be a generic term that includes sustained, delayed, pulsatile, and controlled release.

In another embodiment, the invention is a method for making a composition or formulation, such as a pharmacologic formulation, which method includes dissolving, dispersing, emulsifying, or suspending a BAS, such as an API, in combination with an RR&D and an NVS/P, with or without additional suitable additives. In a preferred embodiment, heat is applied to the RR&D, at or above the rosin softening point, in order to soften it and allow the NVS/P and/or a BAS to be homogeneously blended with it. More than one BAS and/or more than one RR&D and/or more than one NVS/P may be used to make the composition or formulation according to the method of the invention.

In a further embodiment, the invention is a method for making a composition or formulation, such as a pharmacologic formulation, by dissolving, dispersing, emulsifying, or suspending a BAS in a vehicle containing an RR&D and an NVS/P, with or without suitable additives, and by utilizing an additional volatilized organic solvent as a processing aid. For example, it is a common practice in the wet granulation of pharmaceutical tablet production to utilize alcohol as a wetting and/or granulating agent. The alcohol is evaporated from the granulation mixture prior to the formation of tablets. In this case, the alcohol is functioning as a processing aid but is not utilized in the final formation of the formulation. According to this embodiment of the invention, it may be desirable to incorporate a BAS within the RR&D by using a volatilized organic solvent prior to or after combining an NVS/P with the RR&D. The volatilized solvent in this case is utilized as a processing aid and for incorporation of the BAS and to enhance the interaction of the NVS/P and the RR&D. The volatilized solvent is removed from the final dosage form prior to application to the organism or environment and is not utilized as a component of the vehicle.

In another embodiment, the invention is a method of treatment by administering to a patient in need thereof a pharmacological formulation comprising one or more API, one or more NVS/P, and one or more RR&D, with or without suitable additives. According to this embodiment, such administration is preferably oral, rectal, vaginal, parenteral, such as by injection or implantation, or may be by direct application to skin, a mucosal surface, or to a surface of an organ or into a body cavity, such as a naturally occurring body cavity or a cavity created by surgical or non-surgical means, such as by trauma.

The composition of the invention, containing a BAS dissolved, dispersed, emulsified, or suspended within an RR&D-NVS/P vehicle, may be encapsulated, such as within a capsule or within a coating. The capsule or coating compositions may or may not contain additional BAS and may or may not contain an RR&D or an NVS/P. For example, bead or pellet compositions of the invention may be coated to modify the release rate of the BAS, and /or to provide mechanical stability, or to protect the RR&D NVS/P vehicle or vehicle compositions from oxidation.

Such coatings may be made of film-forming polymers such as acrylic polymers, cellulosic polymers, polylactic acid polymers, polyglycolic acid polymers, and co-polymers of polylactic and polyglycolic acid, gelatin, carrageenan, sodium or calcium alginates, agar, gellan, guar, gum arabic, locust bean gum, glucomanan, karaya gum, xanthan, gum tragacanth, polyethylene glycols, carbohydrates and carbohydrate derivatives such as polysaccharides, polyols, dextrins or starches. A plasticizer, such as acetyl triethyl citrate, acetyl tributyl citrate, triethyl citrate, diethyl phthalate, dibutyl phthalate, polyethylene glycol or dibutyl sebacate may be admixed with the polymer of the coating. Methods of making coatings and of coating components of BAS containing compositions are known in the art.

The RR&D of the present invention are insoluble in water at a neutral pH, are biologically compatible, have low toxicity in the levels utilized, may exist as a liquid, semi-solid or solid form, are thermoplastic when in solid form, and are compatible with a wide variety of additional additives and components. One or more suitable RR&D may be utilized within a vehicle to provide the desired release characteristics or processing requirements. An RR&D may also be combined with one or more other RR&Ds of varying physicochemical properties such as different acid number, solubility or different softening temperature.

Preferred rosin resins for the vehicle of the invention are those that have been chemically modified to improve their physical or chemical properties (derivatives). Such modifications may alter softening point, color, acid number, stability, solubility or other chemical and physical characteristics. Examples of suitable derivative modifications include esterification, disproportionation and hydrogenation. Other suitable modifications of rosin resin useful in the invention are found in Modern Technology of Synthetic Resins & Their Applications, Publisher: Asia Pacific Business Press, Authors: National Institute of Industrial Research Board (NIIR BOARD), Delhi India, ISBN: 817833092X pps 503-538.

Preferred rosin resins and derivatives include wood rosin, glycerol ester of wood rosin, pentaerythritol ester of wood rosin, hydrogenated wood rosin, glycerol ester of hydrogenated wood rosin, pentaerythritol ester of hydrogenated wood rosin, and gum rosin and derivatives. The most preferred rosin resin derivatives are esters of rosin, hydrogenated rosin, dimerized/polymerized rosin or disproportionated rosin. The esterification method may vary, however, the modified RR&D should be such that it is compatible with the organism or environment, API and NVS/P utilized it should fall within the scope of the invention. Examples of suitable rosin resin derivatives include those manufactured by Pinova® (Brunswick Georgia) under the Staybelite®, Pentalyn®, Pexite®, and Hercules® brand names. Examples of preferred rosin resins derivatives are Hercules® Ester Gum 8BG glycerol ester of wood rosin. Hercules® Ester Gum 8BG glycerol ester of wood rosin, Hercules® Ester Gum 8BG-HS glycerol ester of wood rosin, Hercolyn® D methyl ester of hydrogenated rosin, Hercules® Ester Gum 8D-A rosin ester, Staybelite® Ester 5 synthetic resin and Staybelite® Ester 5-A synthetic resin. Other preferred examples include polymerized rosin (PR), glycerol ester of polymerized rosin (GPR) and pentaerythritol ester of polymerized rosin (PPR) as manufactured by Derives Resiniques and Terpeniques (DRT), Inc., Gambetta, France.

The vehicle containing the RR&D and NVS/P may be in one of several physical states, depending upon the particular RR&D, or combination of RR&D, or types of NVS/P and/or additives incorporated in the composition containing the vehicle and the indication for which the composition is to be used. For example, the vehicle may be a solid, a liquid, or a semisolid such as a paste, gel, ointment, or lotion. These and other types of pharmacological dosage forms are defined in USP 30 NF 25, Chapter 1151 (2007), First Supplement, which is incorporated herein by reference.

Additionally, additives may be combined with these vehicles to provide compositions that are useful for sustained, delayed, pulsatile, or controlled release indications. These additives may be liquid, solid or semisolid additive components such as polymers, waxes, fillers, antioxidants, colorants, or other pharmaceutical excipients.

In addition to one or more RR&D, the vehicle includes one or more non-volatilized solvents and/or plasticizers (NVS/P)s. The NVS/P may completely or partially solubilize or plasticize just the RR&D, may completely or partially solubilize both the API and the RR&D, or may completely or partially solubilize or plasticize the RR&D, and/or suspend, dissolve or emulsify the API. The NVS/P may also act as a co-solvent or co-plasticizer to another NVS/P. The NVS/P may have variable water solubility or miscibility, a characteristic which may be used to further modulate the release characteristics of the API from the vehicle.

Examples of preferred solvents and plasticizers include but are not limited to citric acid esters such as triethyl citrate (TEC), acetyl triethyl citrate (ATEC), tributyl citrate (TBC), acetyl tributyl citrate (ATBC), acetyltri-n-hexyl citrate (A-6), and butyryltri-triethyl n-hexyl citrate (B-6), lecithin, polyoxyethylene sorbitan fatty acid esters, fatty acid salts, mono and diacetyl tartaric acid esters of mono and diglycerides of edible fatty acids, citric acid esters of mono and diglycerides of edible fatty acids, saccharose esters of fatty acids, polyglycerol esters of fatty acids, polyglycerolesters of internal esterified castor oil acid, sodium stearoyllactylate, polyoxyethylated hydrogenated castor oil, block copolymers of ethylene oxide and propylene oxide, polyoxyethylene fatty alcohol ether, polyoxyethylene stearic acid ester, ethyl lactate, phthalates such as dimethyl phthalate (DMP), diethyl phthalate (DEP), dibutyl phthalate (DBP), dioctyl phthalate, glycol ethers such as ethylene glycol diethyl ether, propylene glycol monomethyl ether, PPG-2 myristyl ether propionate, diethylene glycol monoethyl ether, propylene glycol monotertiary butyl ether, dipropylene glycol monomethyl ether, N-methyl-2-pyrrolidone, 2 pyrrolidone, isopropyl myristate, isopropyl palmitate, octyl palmitate, dimethylacetamide, propylene glycol, propylene glycol monocaprylate, propylene glycol caprylate/caprate, propylene glycol monolaurate, glycerol, glycofurol, linoleic acid, linoeoyl macrogol-6 glycerides, oleic acid and esters such as glyceryl dioleate, ethyl oleate, oleoyl macrogol-6 glycerides, esters such as ethylbenzoate, benzylbenzoate, sucrose esters, sucrose acetate isobutyrate, esters of lactic acid, esters of oleic acid, sebacates such as dimethyl sebacate, diethyl sebacate, dibutyl sebacate, dipropylene glycol methyl ether acetate (DPM acetate), propylene carbonate, propylene glycol laurate, dimethylsulfoxide, polyethylene glycol, dimethyl isosorbide, methylsulfonic acid, gamma butyrolactone, glycerol formal, soketal, ethanol, and water.

Other suitable NVS/P include oils, fats and their derivatives. Oils derived from animals or from plant seeds of nuts typically include glycerides of the fatty acids, chiefly oleic, palmitic, stearic, and linolenic. Non-limiting examples of suitable natural, semi-synthetic and synthetic oils include vegetable oil, peanut oil, medium chain triglycerides, soybean oil, almond oil, olive oil, sesame oil, peanut oil, fennel oil, camellia oil, corn oil, castor oil, cotton seed oil, peppermint oil, safflower oil and soybean oil, either crude or refined, and medium chain fatty acid triglycerides, mineral oils, suitable oil or fat, as for instance completely or partially hydrogenated vegetable oils or completely or partially hydrogenated animal fats, saturated polyglycolized glycerides, semi-synthetic glycerides, glyceryl esters of fatty acids, glyceryl behenate, glyceryl di and tri stearate, glyceryl palmitostearate, lauroyl macrogol-32 glycerides, stearoyl macrogol-32 glycerides, polyethylene glycol esters of fatty acids such as glyceryl laurate, PEG-32 glyceryl palmitostearate, PEG-32 glyceryl stearate, cetyl palmitate, stearyl alcohol, and cetyl alcohol.

Any suitable ratio of RR&D to NVS/P may be utilized to provide the desired release characteristics to the BAS included therein so long as the minimal amount of RR&D in the composition is sufficient to provide a change in the release characteristics of a BAS that is dissolved, dispersed, or suspended in a vehicle containing the RR&D.

For example, the ratio of RR&D to NVS/P may be at any ratio % w/w between 99:1 and 1:99. Specific examples of ratios % w/w of RR&D to NVS/P that may be utilized include 5:95, 10:90, 20:80, 30:70, 40:60, 50:50, 60:40, 70:30, 80:20, 90:10.

### Biologically Active Substance (BAS)

Any chemical compound or combination of chemical compounds that may be dissolved, dispersed, emulsified, or suspended in the vehicle of the invention may be delivered using the vehicle of the invention. Preferably, the substance is a biologically active substance (BAS) and most preferably an active pharmaceutical ingredient (API).

The terms “BAS” and “API” may be used interchangeably in most situations. The use of the term “API” in a particular context within this specification should not be interpreted as being a limitation that excludes non-pharmaceutical BAS. The term biologically active substance (BAS) as used herein refers to molecules including an active pharmaceutical ingredient (API), peptide, protein, carbohydrate (including monosaccharides, oligosaccharides, and polysaccharides), nucleoprotein, mucoprotein, lipoprotein, synthetic polypeptide or protein, or a small molecule linked to a protein, glycoprotein, steroid, nucleic acid (any form of DNA, including cDNA, or RNA, or a fragment thereof), nucleotide, nucleoside, oligonucleotides (including antisense oligonucleotides), gene, lipid, hormone, vitamin, including vitamin C and vitamin E, or combination thereof, that causes a biological effect when administered to an animal, including but not limited to birds and mammals, including humans. The term biologically active substance also includes agents such as insecticides, pesticides, cosmetic active agents, fungicides, rodenticides, and plant nutrients and growth promoters.

The term active pharmaceutical ingredient (API), as used herein, refers to any substance used internally or externally as a medicine for the treatment, cure, diagnosis or prevention of a disease or disorder, and includes but is not limited to immunosuppressants, analgesics, respiratory agents, antioxidants, anesthetics, chemotherapeutic agents, steroids (including retinoids), hormones, antibiotics, antivirals, antifungals, antiproliferatives, antihistamines, anticoagulants, antiphotoaging agents, melanotropic peptides, nonsteroidal and steroidal anti-inflammatory compounds, antipsychotics, and radiation absorbers, including UV-absorbers.

Particular APIs that are suitable for the invention include, but are not limited to, anticancer agents such as taxol, carmustine, interleukin 2, and interferon; growth hormones such as human growth hormone and somatotropin hormone; antipsychotic agents such as risperidone, and fluphenazine decanoate; antibiotics such as gentamicin, tetracycline, oxytetracycline, cephalosporins, aminoglycosides, and sulfonamides; oxytocic agents and prostaglandins; topical anesthetic agents such as benzocaine, chloroprocaine, cocaine, procaine, propoxycaine, tetracaine, depravaine, bupivacaine, etidocaine, levobupivacaine, lidocaine, mepivacaine, prilocaine, propofol and ropivacaine; systemic analgesic agents such as opioids or other narcotics, including morphine, oxycodone, hydrocodone, oxymorphone, hydromorphone, tramadol, fentanyl, sufentanyl, and butorphanol; narcotic antagonists such as naltrexone, nalorphine, naloxone, and nalmefene; growth promoting agents such as TGF alpha and TGF beta; bone morphogenic peptides and proteins and calcium salts such as calcium sulfate, calcium phosphate; anti-inflammatory agents such as diclofenac; steroids such as protaglandins, estrogens, androgens, and progestins; acne medications such as benzoyl peroxide; hair conditioners; sun screens; medications for skin conditions such as zinc oxide and those for treatment of psoriasis; ophthalmics such as lubricants and anti-glaucoma therapeutics; antibiotics such as quinolones; saliva substitutes; sedative/hypnotics such as benzodiazepines and barbiturates; wound care such as growth factors (EPO, FGF, G-CSF); antiparasitics for the treatment of diseases such as worms and malarial infestations; anticonvulsants; muscle relaxants; nucleoside analogs; osteoporosis preparations, such as to supplement bone growth; and antiparkinsonian agents and respiratory agents such as dextromethorphan, pseudoephederine, phenylepherine, guaifenesin, and carbinoximine.

The API may be present in any physical state, such as a solid, a liquid, or a semisolid. Solid API may be crystalline or amorphous, or a combination thereof. Such solid API may be granulated with or without added excipients, and may be encapsulated in a material such as a polymer and/or a wax. A solid API may also be in the form of a matrix in which the API is distributed therein. A liquid API may be granulated, such as by absorption to a solid substrate, or encapsulated with a suitable solid, such as a polymer or wax or combination thereof or blended with the vehicle in its liquid state.

Generally, a composition that is hydrophilic will tend to release BAS such as an API loaded in the vehicle more rapidly than a similar formulation possessing hydrophobic properties. Also, a more viscous composition may release BAS at a slower rate than a similar less viscous composition. Additionally, a vehicle constituting a RR&D and NVS/P and combination of hydrophilic and hydrophobic components or additives may modulate the release characteristics of the dissolved, dispersed, emulsified, or suspended BAS from the compositions.

Thus, the release characteristics and absorption of a BAS may be modulated by the rheology and/or hydrophilicity or hydrophobicity of the vehicle containing the RR&D and NVS/P. The release characteristics may be further modified by combining one or more suitable additives in or with the vehicle. These additives may include additional RR&Ds and NVS/Ps of varying physicochemical, hydrophilic or hydrophobic characteristics. The release characteristics and absorption of a BAS may be further modified by modulating the ratio of RR&D to NV S/P and the amount of BAS incorporated in the composition.

Thus, controlled or modified release of a BAS from the vehicle containing an RR&D and an NVS/P may be obtained by the use of one RR&D and one NVS/P as the vehicle or by combining one or more RR&Ds with different physical and chemical properties, one or more NVS/P with different physical and chemical properties, and/or by optionally combining one or more additives of different physical and chemical properties.

Additives may be included in the composition containing the vehicle of the invention in order to obtain the desired release characteristics of the API. Liquid, semisolid, or solid additives may be added, either singly or in combination, to the vehicles to modify the physicochemical as well as biological characteristics of the vehicle such as, hydrophilicity or hydrophobicity, consistency or viscosity, absorption rate and degradation rate at implantation or application sites, color, and stability. Addition of hydrophilic liquid, semisolid, or solid additives will increase the hydrophilicity of the vehicle prepared from blends of RR&D and NV S/P whereas, addition of hydrophobic liquid, semisolid or solid additives will increase the hydrophobicity of a vehicle prepared from blends of RR&D NVS/P. Hydrophilic vehicles may tend to be absorbed more rapidly or release the BAS more rapidly than the hydrophobic vehicles from the site of administration, injection or application. Addition of semisolid and solid additives may increase the viscosity of the vehicles, which generally decreases the release rate of a BAS as compared to addition of a liquid additive. By way of example, it is possible to extend the in vivo duration of stay of an implanted or an injected vehicle prepared from blends of RR&D and NVS/P by adding a hydrophobic wax or other hydrophobic solid additives, which will increase both hydrophobicity and viscosity of the vehicle. The physical state of the vehicle may thus be liquid, semisolid, gel, paste, or solid. Depending on the intended use and the components of the vehicle, the formulation containing the vehicle may be altered to obtain the desired release characteristics for the API. The amount of additive used will in general be a function of the nature of the additive and the effect to be achieved, and can be easily determined by the practitioner.

### Additives

A variety of additives may be optionally added to the vehicle or composition, so as to obtain a vehicle or composition with desired properties and/or API release characteristics. The additives may also be used as processing aids, and thus facilitate the formation of the vehicle or composition. The additives may be present in any amount which is sufficient to impart the desired properties to the vehicle or the compositions. The amount of additive used will in general be a function of the nature of the additive and the effect to be achieved, and can be easily determined by one skilled in the art.

When present, the additive is typically present in the compositions in an amount ranging from about 0.1 percent to about 99 percent by weight, relative to the total weight of the composition, and more typically, is present in the composition in an amount ranging from about 1, 2, or 5 percent to about 40 percent by weight. Certain additives, such as buffers, are only present in small amounts in the composition while certain polymers may be present at higher levels, such as 20 to 30 percent

The following categories are non-limiting examples of classes of additives that may be employed in the composition. Given the disclosure herein and the objects to be achieved, one of skill in the art will easily know how to select other additives to achieve a desired purpose. All of these embodiments are considered to fall within the disclosed invention.

### A. Polymers

One category of additives is polymers, copolymers and oligomers. The polymers may be used to alter the release profile of the substance to be delivered, to add integrity to the composition, or to otherwise modify the physicochemical properties including hydrophilicity and hydrophobicity of the composition. Non-limiting examples of suitable polymers, copolymers and oligomers include polyacrylates, ethylene-vinyl acetate polymers, cellulose and cellulose derivatives, such as hydroxyethylcellulose, hydroxypropylcellulose, hydroxypropylmethylcellulose, methyl cellulose, sodium carboxymethylcellulose cellulose esters, cellulose acetate, acyl substituted cellulose acetates and derivatives thereof, non-erodible polyurethanes, polystyrenes, polyvinyl chloride, polyvinyl fluoride, poly(vinyl imidazole), chlorosulphonated polyolefins, polyethylene oxide, poly(lactide), poly(lactide-co-glycolide), poly(glycolide), poly(caprolactone), poly (vinyl alcohol), poly(epsilon-caprolactone), poly(delta-valerolactone) and poly(gamma-butyrolactone), polyamides, polyanhydrides, polyamino acids, polyorthoesters, polycyanoacrylates, poly(phosphazines), poly(phosphoesters), polyesteramides, polydioxanones, polyacetals, polyketals, polycarbonates, polyorthocarbonates, degradable polyurethanes, polyhydroxybuty-ates, polyhydroxyvalerates, polyalkylene oxalates, polyalkylene succinates, poly(malic acid), polyethylene, polyvinyl pyrrolidone, ethylene vinylacetate, polyethylene glycol, cellulose acetate, (CE), cellulose acetate butyrate (“CAB”), cellulose acetate propionate (“CAP”), chitin, chitosan, and copolymers, terpolymers, oxidized cellulose or combinations, chemically modified, isomers, or mixtures of the above materials.

### B. Waxes and Their Derivatives.

Waxes include those derived from animal, seeds, flowers, fruits, leaves, stem or any part of a plant or tree, hydrogenated oils, natural, semi-synthetic or synthetic waxes, castor wax, microcrystalline wax , rice bran wax , carnauba wax, beeswax, paraffin, stearic acid, salts of stearic acid, cetyl alcohol, cholesterol, derivatives of beeswax and camauba wax, candelilla wax, cocoa butter, and degreased cocoa powder.

### C. Carbohydrates and Carbohydrate Derivatives

Non-limiting examples of carbohydrates and carbohydrate derivatives compounds include monosaccharides (simple sugars such as fructose and its isomer glucose (dextrose); disaccharides such as sucrose, maltose, cellobiose, lactose; starch; polysaccharides; polyols such as mannitol and sorbitol; dextrins such as maltodextrin, and cyclodextrins such as α-cyclodextrin, β-cyclodextrin, hydroxypropyl-β-cyclodextrin, and sulfobutylether-β-cyclodextrin.

### D. Additional Additives

Other components, such as preservatives, stabilizers, anti-oxidants, coloring agents, isotonic agents, flavorings, humectants, sequesterants, vitamins and vitamin precursors, salts, surfactants, and phospholipids may be added as desired. As preferred examples of preservatives, paraben derivatives are given with methyl paraben and propyl paraben given as most preferred preservatives. As preferred examples of anti-oxidants, butyl hydroxyanisole, butyl hydroxytoluene, propyl gallate, vitamin E acetate, and purified hydroquinone are given with vitamin E acetate and butyl hydroxytoluene given as most preferred anti-oxidants. Given as preferred examples of humectant is sorbitol. Given as preferred examples of flavorings are peppermint oil, spearmint oil, wintergreen oil, menthol and saccharin. Given as a preferred example of a sequesterant is citric acid. Preferred examples of vitamins are vitamin A, C, D and E and K, and vitamin E acetate. Examples of salts include aluminum salts, aluminum monostearate, magnesium hydroxide, and aluminum hydroxide, zinc salts, tannic acid salts, salts of acids and bases such as sodium and potassium phosphates, sodium and potassium hydroxide, sodium and potassium carbonates and bicarbonates. Preferred surfactants are non-ionic surfactants, such as Cremophor EL, Cremophor RH 40, Cremophor RH 60, polyethylene glycol 1000 succinate, polysorbate 20, polysorbate 80, Solutol HS 15, sorbitan monooleate, poloxamers such as polaxamer 188, Labrafil M-1944CS, Labrafil M-2125CS, Labrasol, Gellucire 44/14, and Softigen 767. Examples of preferred phospholipids are hydrogenated soy phosphatidylcholine, distearoylphosphatidylglycerol, 1-α-dimyristoylphosphatidylcholine, and 1-α-dimyristoylphosphatidylglycerol.

In addition to the above ingredients, a composition containing the RR&D NVS/P vehicle may contain suitable quantities of other materials such as lubricants, binders, granulation aids, diluents and glidants that are conventional in the pharmaceutical art.

The composition of the invention containing one or more API and a vehicle containing one or more RR&D and NVS/P may be prepared by any suitable blending or incorporation method. Typically, the RR&D is heated to or above its softening point and the NVS/P is added until the vehicle achieves a uniform consistency and desired rheological characteristics. The API is then added either at the same or a higher or lower temperature and combined with the vehicle, such as by blending or mixing until the API is dissolved, or dispersed, emulsified, or suspended in the vehicle. The API may be added at any stage in the preparation. For example, the API may be combined with the RR&D before the NVS/P is combined, or may be combined with the NVS&P before the RR&D is combined, or may be combined with the RR&D and NVS/P following the combination of these components.

Additives, such as those described above, may be combined with the API and the vehicle before, during, or after the combining of the API and the vehicle. It may be further desirable to solubilize the RR&D with an organic volatilized solvent and techniques such as described in Lee et al, Rosin nanoparticles as a drug delivery carrier for the controlled release of hydrocortisone, Biotechnology Letters (2005) 27:1487-1490 and in Sheomey and Dorle, Effects of solvents on the characteristics of rosin walled microcapsules prepared by solvent evaporation technique, J. Microencapsulation, 1991, Vol. 8, No. 1, 71-7, prior to incorporating the API and NVS/P. It is intended that the volatilized solvent is being used as a processing aid in the manufacturing of the vehicle and is not present at all or in insignificant quantities in the final vehicle composition.

The composition of the invention may be in any suitable form such as granules, spheres, beads, pellets, rods, implants, lotions, gels, liquids, and pastes. The vehicle of the invention may be a part of a composition that is filled into a hard or soft shell capsule or sachet or that is compressed, extruded, or otherwise formed into tablets. The vehicle or composition containing the vehicle may be delivered via any suitable method such as a patch, a semisolid such as a gel, ointment or paste for administration to the skin or mucous membrane, shaped into particulates such as but not limited to granules, rods, cylinders, beads or pellets for oral or parenteral administration or made into a tablet for oral administration. The particulates may be filled into capsules. Alternatively, the vehicle containing the API may be placed into a secondary delivery system for injection or implantation under the skin or placed into a cavity of the body, such as those due to surgical or non-surgical trauma.

The vehicle or composition of the invention may be in the form of a solid, a semi-solid, paste, gel, or a liquid. The forms of a semi-solid, paste, gel, or liquid are especially useful to be filled into capsule dosage forms, such as a soft or hard gelatin capsules or formed into suppositories. Solid forms include microcapsules, microspheres, powders, granules and beads. Methods of preparing microcapsules, microspheres, powders, granules, and beads are well known in the art and include but are not limited to spray drying, spray chilling, rotary disk atomization, fluid bed coating, stationary nozzle coextrusion, centrifugal head coextrusion, and submerged nozzle coextrusion, extrusion-spheronization, hot-melt extrusion, phase separation, solvent evaporation, solvent extraction, interfacial polymerization, simple and complex coacervation and in-situ polymerization.

According to the method of treatment embodiment of the invention, a medical condition is sought to be prevented, treated, diagnosed or mitigated by administering to a patient a pharmaceutical formulation containing a composition of the invention as described above. The administration may be internal, such as oral or parenteral, such as internal parenteral administration including but not limited to intravascular, intramuscular, subcutaneous, intradermal, intrathecal, and intracavitary routes of administration, as well as application to the external surface of an internal bodily organ, such as during a surgical or laparoscopic procedure. The administration may be topical, including administration to the skin or to a mucosal surface, including the oral, vaginal, rectal surfaces, to the surface of the eye, to the nasal passages, or to the ear canal. The RR&D formulation can be used to deliver BAS to other animal species besides humans, such as for veterinary purposes, or to the environment, such as for agriculture purposes.

The invention is further illustrated in the following non-limiting examples.

### EXAMPLE 1

### Compositions Containing RR&D, NVS/P, and BAS

Compositions were prepared containing guaifenesin (BAS) blended into a vehicle containing a glycerol ester of wood rosin (RR&D) and triethyl citrate (TEC) (NVS/P) as shown in Table 1.

TABLE 1

| Ingredients | GFN2 (% w/w) | GFN16 (% w/w) | GFN18 (% w/w) |
|---|---|---|---|
| Guaifenesin | 50.0 | 55.0 | 40.0 |
| (BAS) | | | |
| Glycerol Ester of | 40.0 | 31.0 | 39.0 |
| Wood Rosin | | | |
| (RR&D) | | | |
| Triethyl Citrate | 10.0 | 14.0 | 21.0 |
| (TEC), (NVS/P) | | | |

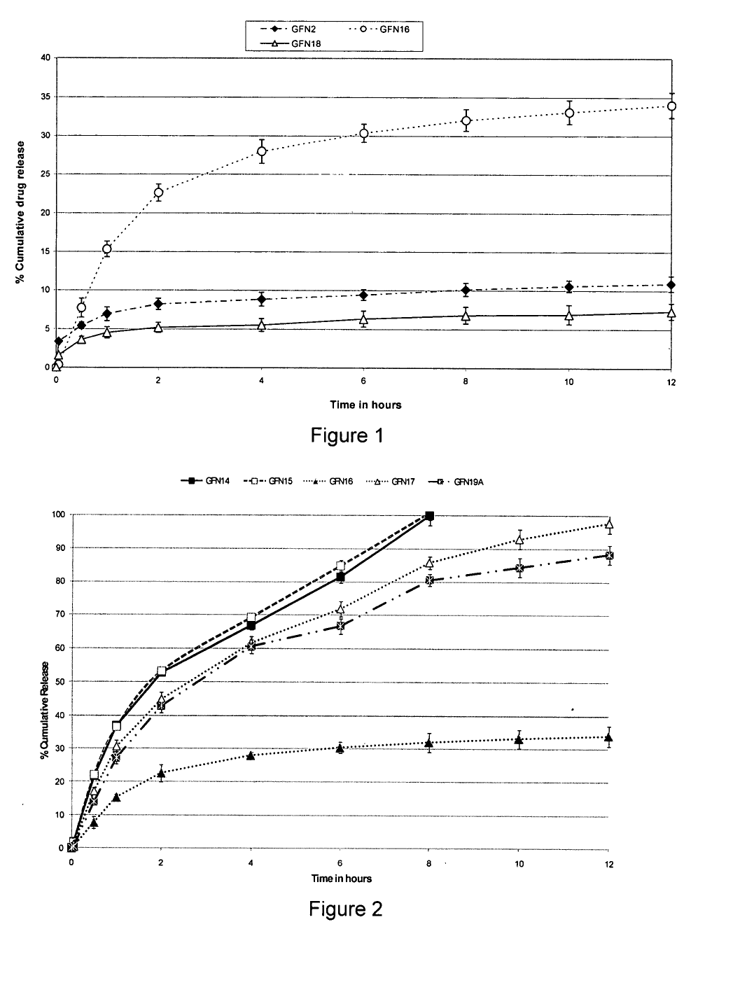

The BAS was passed through an 80-mesh screen. Weighed quantities of the RR&D and the NVS/P were heated together in a porcelain vessel. Heating was continued until the RR&D was fluid. The resulting molten mass was allowed to cool under continuous mixing. The screened guaifenesin was then added to the RR&D-NVS/P gelling mass gradually and under continuous mixing at about 60 to 70° C. During the addition of the BAS, intermittent heating of the gelling mass was carried out to maintain the temperature at 60 to 70° C. After completion of BAS addition, mixing was continued further for a period of 30-45 minutes as the gel mass cooled down to room temperature to obtain the BAS-loaded vehicle. BAS release from the BAS-loaded vehicle was determined using a USP Type II apparatus at 50 RPM with 900 ml of water as the dissolution medium. Sampling intervals were 0.5, 1.0, 2.0, 4.0, 6.0, 8.0, 10.0 and 12.0 hours. Quantification of the drug in the dissolution samples was performed using ultra-violet spectrophotometry. The results of the dissolution study are illustrated in FIG. 1.

As shown in FIG. 1, the release of the BAS from the composition may be varied by altering the relative concentrations of RR&D and NVS/P in the vehicle of the composition.

### EXAMPLE 2

### Compositions Containing a Multiplicity of NVS/P

Compositions were prepared containing guaifenesin (BAS) blended into a vehicle containing a glycerol ester of wood rosin (RR&D) and a multiplicity of NVS/P (triethyl citrate (TEC) and polyethylene glycol (PEG 6000)), as shown in Table 2. The compositions were prepared according to the method of Example 1.

TABLE 2

| | | | | GFN17 | |
|---|---|---|---|---|---|
| | GFN14 | GFN15 | GFN16 | (% | GFN19A |
| Ingredients | (% w/w) | (% w/w) | (% w/w) | w/w) | (% w/w) |
| Guaifenesin | 55.0 | 60.0 | 55.0 | 55.0 | 55.0 |
| (BAS) | | | | | |
| Glycerol Ester | 18.0 | 20.0 | 31.0 | 27.0 | 28.5 |
| of Wood Rosin | | | | | |
| (RR&D) | | | | | |
| Triethyl Citrate | 13.5 | 12.0 | 14.0 | 12.0 | 13.5 |
| (TEC), | | | | | |
| (NVS/P) | | | | | |
| polyethylene | 13.5 | 8.0 | 0.0 | 6.0 | 3.0 |
| glycol 6000 | | | | | |
| (PEG 6000), | | | | | |
| (NVS/P) | | | | | |

BAS release from the BAS-loaded vehicle was determined as in Example 1. The results of the dissolution study are illustrated in FIG. 2.

As shown in FIG. 2, the release of the BAS from the composition may be varied by altering the relative concentrations of RR&D and NV S/P in the vehicle of the composition and the relative concentrations of the components of the vehicle.

### EXAMPLE 3

### Compositions Containing a Multiplicity of NVS/P

Compositions were prepared according to the method of Example 1, except that guaifenesin (BAS) was blended into a vehicle containing a glycerol ester of wood rosin (RR&D) and a multiplicity of NVS/P (selected from triethyl citrate (TEC), acetyltriethyl citrate (ATEC), and polyethylene glycol (PEG 6000)), as shown in Table 3. The concentrations of BAS and RR&D were the same in each of the compositions of Table 3, as were the total concentrations of NVS/P. The relative concentrations of the various NVS/P of the vehicle were altered amongst the compositions.

TABLE 3

| | GFN17 | GFN19B | GFN20A |
|---|---|---|---|
| Ingredients | (% w/w) | (% w/w) | (% w/w) |
| Guaifenesin (BAS) | 55.0 | 55.0 | 55.0 |
| Glycerol Ester of | 27.0 | 27.0 | 27.0 |
| Wood Rosin | | | |
| (RR&D) | | | |
| Triethyl Citrate | 12.0 | 6.0 | 0.0 |
| (TEC), (NVS/P) | | | |
| Acetyltriethyl | 0.0 | 6.0 | 12.0 |
| Citrate | | | |
| (ATEC), (NVS/P) | | | |
| Polyethylene | 6.0 | 6.0 | 6.0 |
| Glycol 6000 (PEG | | | |
| 6000), (NVS/P) | | | |

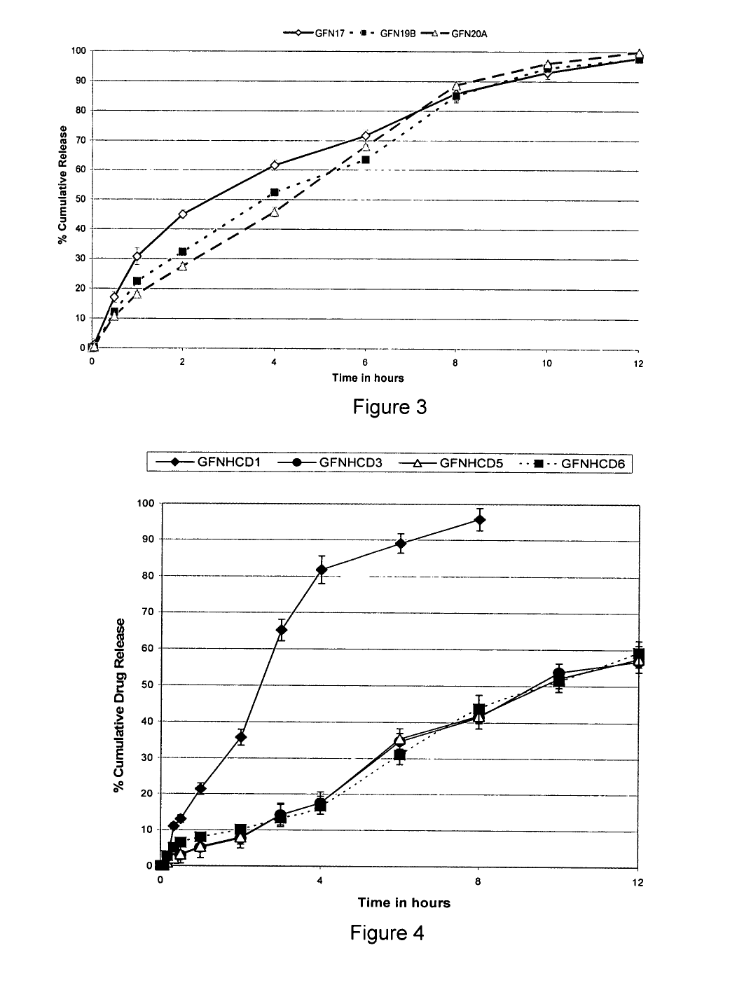

BAS release from the BAS-loaded vehicle was determined as in Example 1. The results of the dissolution study are illustrated in FIG. 3.

As shown in FIG. 3, the release of the BAS from the composition may be varied by altering the constituents of the NVS/P component of the vehicle, even while maintaining the total concentration of NVS/P of the vehicle constant.

### EXAMPLE 4

### Compositions Containing a Multiplicity of BAS and a Multiplicity of NVS/P

Compositions were prepared according to the method of Example 1, except that a multiplicity of BAS, guaifenesin and hydrocodone, was blended into a vehicle containing a glycerol ester of wood rosin (RR&D) and a multiplicity of NVS/P (acetyltriethyl citrate (ATEC) and polyethylene glycol (PEG 6000), as shown in Table 4, and the method of quantification was by high performance liquid chromatography with ultraviolet detection (HPLC with UV detection). The guaifenesin was passed through an 80-mesh screen and the hydrocodone was passed through a 400-mesh screen. The guaifenesin and hydrocodone were mixed geometrically, prior to adding to the RR&D-NVS/P gelling mass.

TABLE 4

| | GFNHCD1 | GFNHCD3 | GFNHCD5 | GFNHCD6 |
|---|---|---|---|---|
| Ingredients | (% w/w) | (% w/w) | (% w/w) | (% w/w) |
| Guaifenesin | 55.0 | 45.0 | 44.26 | 49.18 |
| (GGE), (BAS) | | | | |
| Hydrocodone | 0.92 | 0.75 | 0.74 | 0.82 |
| Bitartrate (HC), | | | | |
| (BAS) | | | | |
| Glycerol Ester | 26.4 | 33.0 | 33.01 | 30.0 |
| of Wood Rosin | | | | |
| (RR&D) | | | | |
| Acetyltriethyl | 11.68 | 15.25 | 14.66 | 13.34 |
| Citrate | | | | |
| (ATEC), | | | | |
| (NVS/P) | | | | |
| Polyethylene | 6.0 | 6.0 | 7.33 | 6.67 |
| Glycol 6000 | | | | |
| (PEG 6000), | | | | |
| (NVS/P) | | | | |

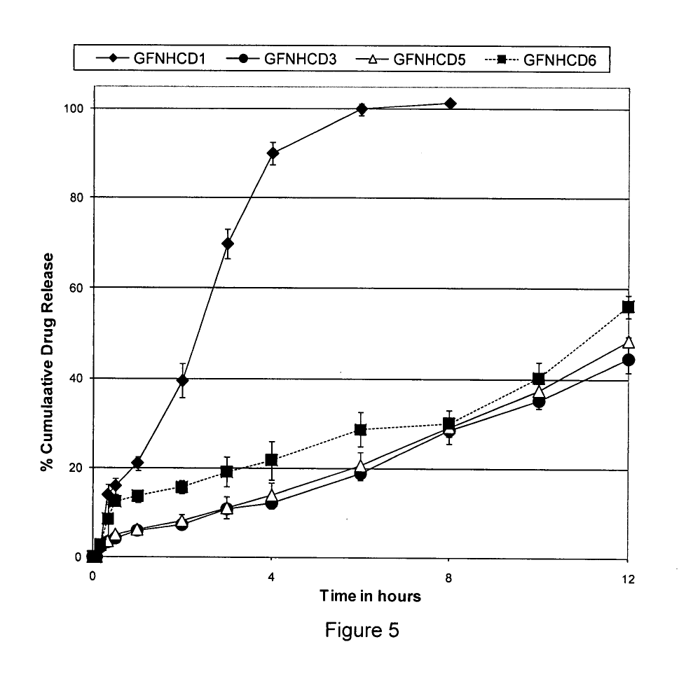

BAS release from the BAS-loaded vehicle was determined as in Example 1. The results of the dissolution study are illustrated in FIGS. 4 and 5.

As shown in FIG. 4, the release profile of the BAS guaifenesin from three of the compositions shown in Table 4 was approximately the same. However, the release from composition GFNHCD1 that contained a higher proportion of BAS and a lower proportion of both the RR&D and NVS/P was much more rapid. FIG. 5 shows a release profile of the BAS hydrocodone from the compositions similar to that of the BAS guaifenesin. It is noted that guaifenesin and hydrocodone have similar solubility in water.

### EXAMPLE 5

### Composition Containing a Multiplicity of BAS and a Multiplicity of NVS/P

A composition was prepared according to Example 5 as shown in Table 5.

TABLE 5

| | GFNHCD10 |
|---|---|
| Ingredients | (% w/w) |
| Guaifenesin (GGE), (BAS) | 49.18 |
| Hydrocodone Bitartrate | 0.82 |
| (HC), (BAS) | |
| Glycerol Ester of Wood | 28.57 |
| Rosin (RR&D) | |
| Acetyltriethyl Citrate | 13.33 |
| (ATEC), (NVS/P) | |
| Polyethylene Glycol 6000 | 8.09 |
| (PEG 6000), (NVS/P) | |

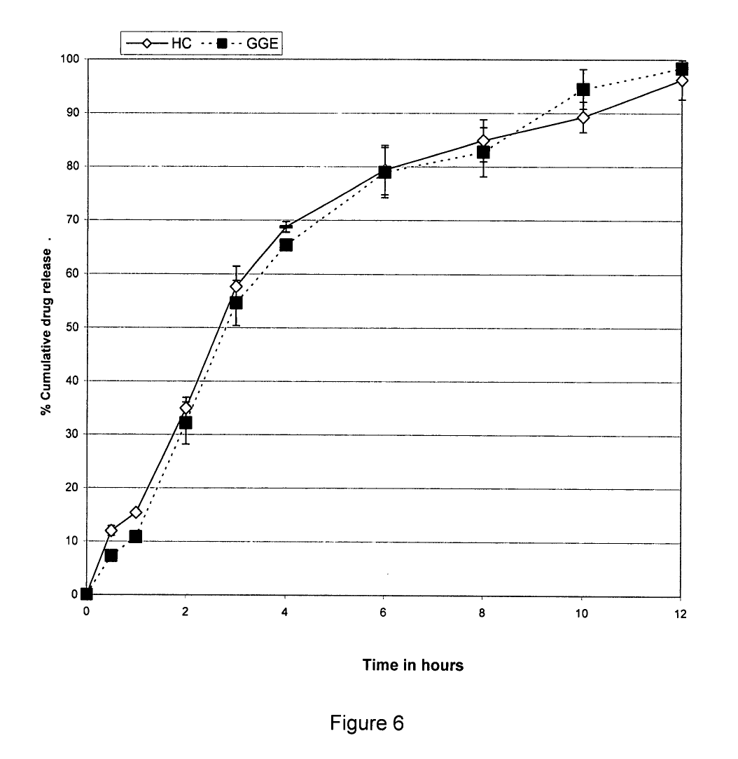

Release of each BAS from the loaded vehicle was determined as in Example 1. The results of the dissolution study are illustrated in FIG. 6.

As shown in FIG. 6, the release profiles of the BAS guaifenesin and of the BAS hydrocodone from the composition of Table 5 were approximately the same.

### EXAMPLE 6

### Compositions Containing a Multiplicity of NVS/P

Compositions were prepared according to the method of Example 1, except that guaifenesin (BAS) was blended into a vehicle containing a glycerol ester of wood rosin (RR&D) and either a single NVS/P or a multiplicity of NVS/P (tetraglycol with or without polyethylene glycol (PEG 6000)) as shown in Table 6. Two of the compositions contained 10% PEG in addition to varying concentrations of tetraglycol and two of the compositions contained no PEG and had varying concentrations of tetraglycol. The concentration of RR&D was varied to maintain the same ratio of BAS to vehicle of 25:75.

TABLE 6

| | GFN52 | GFN53 | GFN54 | GFN55 |
|---|---|---|---|---|
| Ingredients | (% w/w) | (% w/w) | (% w/w) | (% w/w) |
| Guaifenesin | 25.0 | 25.0 | 25.0 | 25.0 |
| (BAS) | | | | |
| Glycerol Ester of | 47.5 | 52.5 | 67.5 | 63.8 |
| Wood Rosin | | | | |
| (RR&D) | | | | |
| Polyethylene | 10.0 | 10.0 | — | — |
| Glycol 6000 (PEG | | | | |
| 6000), (NVS/P) | | | | |
| Tetraglycol | 17.5 | 12.5 | 7.5 | 11.25 |
| (NVS/P) | | | | |

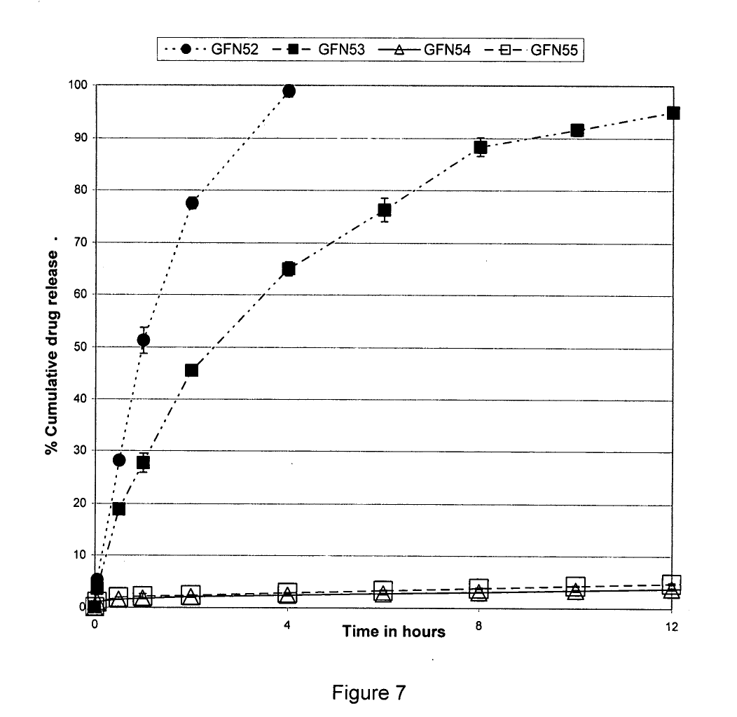

Release of BAS from each of the loaded vehicles was determined as in Example 1. The results of the dissolution study are illustrated in FIG. 7.

As shown in FIG. 7, the percentage of guaifenesin released from compositions containing vehicles with PEG was more rapid than from compositions lacking PEG.

### EXAMPLE 7

### Effect of RR&D on Drug Release Rate

Compositions containing a constant concentration of a BAS and varying concentrations of an RR&D and an NVS/P were made in order to demonstrate the effect of the presence of an RR&D on release rate of the BAS from the compositions. The compositions are shown in Table 7. The BAS (guaifenesin) was passed through an 80-mesh screen. For compositions GFN 70, 71, and 72, weighed quantities of RR&D (Ester Gum 8BG) were heated past the softening point in a porcelain vessel. Screened BAS was then added to the RR&D gelling mass gradually and under continuous mixing at about 60 to 70° C. During BAS addition, intermittent heating of the gelling mass was carried out to maintain the temperature at 60 to 70° C. After completion of BAS addition, mixing was continued further for a period of 30-45 minutes as the gel mass cooled down to room temperature. The resultant solidified RR&D-BAS mass was crushed to powder and passed through 80 mesh sieve. The powdered RR&D-BAS mass was dispersed in a weighed quantity of NVS/P by gradual addition under continuous mixing to obtain a BAS loaded vehicle.

For composition GFN 69, which contained no RR&D, a weighed quantity of NVS/P was added to a vessel. Screened BAS was then added to the NVS/P under continuous mixing to form a NVS/P-BAS composition.

TABLE 7

| | GFN69 | GFN71 | GFN70 | GFN72 |
|---|---|---|---|---|
| Ingredients | (% w/w) | (% w/w) | (% w/w) | (% w/w) |
| Guaifenesin | 50.0 | 50.0 | 50.0 | 50.0 |
| (BAS) | | | | |
| Glycerol Ester of | 0.0 | 2.0 | 5.0 | 10.0 |
| Wood Rosin | | | | |
| (RR&D) | | | | |
| Triethyl Citrate | 50.0 | 48.0 | 45.0 | 40.0 |
| (TEC), (NVS/P) | | | | |

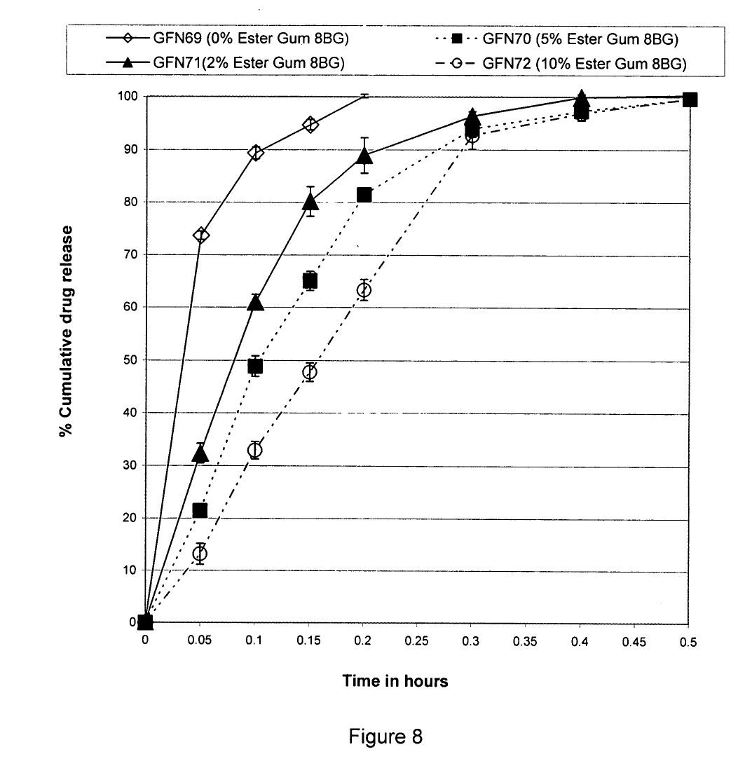

Release of BAS from each of the compositions of Table 7 was determined using a USP Type II apparatus at 50 RPM with 900 ml of water as the dissolution medium. Sampling intervals were 0, 0.05, 0.1, 0.15, 0.2, 0.3, 0.4, and 0.5 hours. Quantification of the BAS in the dissolution samples was performed using ultra-violet spectrophotometry. Results are shown in FIG. 8.

As shown in FIG. 8, the RR&D slowed release of the BAS from the compositions in a concentration-dependent manner. FIG. 8 further shows that even very low concentrations of RR&D are effective in modifying the release rate of a BAS from a composition compared to a similar composition lacking an RR&D.

Further modifications, uses, and applications of the invention described herein will be apparent to those skilled in the art. It is intended that such modifications be encompassed within the scope of the following claims.

1. A delivery vehicle for a biologically active substance (BAS) comprising a rosin resin or derivative (RR&D) and a non-volatilized solvent or plasticizer (NVS/P), wherein the concentration of the RR&D relative to the NVS/P in the delivery vehicle is sufficient to modify the rate of release of a BAS from the delivery vehicle as compared to the rate of release of the BAS from a similar delivery vehicle that is free of the RR&D.

2. The delivery vehicle of claim 1 wherein the ratio of concentrations % w/w of the RR&D and the NVS/P is at least 4:96.

3. The delivery vehicle of claim 2 wherein the ratio is at least 10:90.

4. The delivery vehicle of claim 2 wherein the ratio is at least 20:80.

5. The delivery vehicle of claim 1 wherein the concentration of RR&D in the vehicle is higher than the concentration of the NVS/P.

6. The delivery vehicle of claim 1 in which a BAS is dissolved, dispersed, suspended, or emulsified.

7. The delivery vehicle of claim 1 which further comprises a polymeric additive.

8. A composition for delivery of a biologically active substance (BAS), comprising the BAS and a delivery vehicle comprising a rosin resin or derivative (RR&D) and a non-volatilized solvent and/or plasticizer (NVS/P), wherein the BAS is dissolved, dispersed, suspended, or emulsified in the delivery vehicle, and wherein the concentration of the RR&D in the delivery vehicle is sufficient to modify the rate of release of a BAS from the composition as compared to the rate of release of the BAS from a similar composition containing a delivery vehicle that is free of the RR&D.

9. The composition of claim 8 wherein the ratio of the RR&D:NVS/P in the vehicle is at least 4:96.

10. The composition of claim 9 wherein the ratio is at least 10:90.

11. The composition of claim 9 wherein the ratio is at least 20:80.

12. The composition of claim 8 wherein the concentration of RR&D in the vehicle is higher than the concentration of the NVS/P.

13. The composition of claim 8 which further comprises a polymeric additive and which composition is in the form of a semisolid.

14. The composition of claim 8 which is in the form selected from the group consisting of liquid, semisolid, solid, gel, paste, ointment, and lotion.

15. The composition of claim 9 wherein, upon administration, the BAS is released from the composition in a sustained, pulsatile, or controlled manner.

16. The composition of claim 9 wherein the BAS is an analgesic medication.

17. The composition of claim 16 wherein the analgesic medication is a narcotic.

18. A method for making a composition for delivery of a biologically active substance (BAS) comprising blending a rosin resin or derivative (RR&D) and a non-volatilized solvent and/or plasticizer (NVS/P) and dissolving, dispersing, emulsifying, or suspending the BAS in the blended RR&D and NVS/P.

19. The method of claim 18 which comprises combining a volatilized solvent with the RR&D before blending the RR&D and the NVS/P and then removing the volatilized solvent to obtain the composition.

20. The method of claim 19 wherein all of the volatilized solvent that was combined with the RR&D is removed.

21. A method for administering or applying a controlled release formulation of a biologically active substance (BAS) comprising obtaining a formulation that contains a composition which comprises a BAS and a delivery vehicle comprising a rosin resin or derivative (RR&D) and a non-volatilized solvent or plasticizer (NVS/P), wherein the concentration of the RR&D relative to the NVS/P in the delivery vehicle is sufficient to modify the rate of release of a BAS from the delivery vehicle as compared to the rate of release of the BAS from a similar delivery vehicle that is free of the RR&D, and administering the formulation to an individual in need thereof or applying the formulation to the environment.

22. The method of claim 21 wherein the BAS is an active pharmaceutical ingredient (API) and the formulation is administered to an individual.

23. The method of claim 21 wherein the ratio of the RR&D:NVS/P in the vehicle is at least 4:96.

24. The method of claim 23 wherein the ratio is at least 10:90.

25. The method of claim 23 wherein the ratio is at least 20:80.

26. The method of claim 13 wherein the concentration of RR&D in the vehicle is higher than the concentration of the NVS/P.
